(19) United States
(12) Patent Application Publication
(10) Pub. No.: US 2017/0028169 A1
(43) Pub. Date: Feb. 2, 2017

(54) HYDROPHOBIC TEXTURISED FOLEY CATHETER WITH LOW MICROBIAL ADHESION

(71) Applicant: EQUIPOS MEDICOS VIZCARRA, S.A., Emiliano Zapata, Morelos (MX)

(72) Inventors: Jose Antonio Arellano Cabrera, EMiliano Zapata (MX); Jose Maria Rodriguez Lelis, Emiliano Zapata (MX); Maria Teresa Lucas Jimenez, Emiliano Zapata (MX); Alfredo Chalita Vizcarra, Emiliano Zapata (MX)

(21) Appl. No.: 15/302,265

(22) PCT Filed: Apr. 6, 2015
(86) PCT No.: PCT/MX2015/000058

§ 371 Date: Oct. 6, 2016

(30) Foreign Application Priority Data
Apr. 8, 2014 (MX) MX/A/2014/004232

(51) Int. Cl. A61M 25/00

(52) U.S. Cl. CPC A61M 25/0017

(57) ABSTRACT

The invention relates to medical equipment for treatment, specifically medical equipment for extracting and draining fluids contained in the bladder of a patient. The Foley catheter according to the invention comprises tubing, with at least two lumens, made of a biocompatible polymer material with a length of between 100 and 400 mm and outer diameters of between 1 and 15 mm. The surface of the tubing has a high-relief texture with a maximum height of 30 micrometers and patterns designed to generate the property of hydrophobicity and to reduce the adhesion of bacteria to said surface. A collecting funnel with at least two channels, made from medical-grade polymer material, is fixed to the distal end of the tip of the tubing, while the tip of the tubing is closed in such a way that it forms a rounded tip. A flexible inflatable balloon is arranged upstream of the tip, for the anchoring of the catheter.

### FIELD OF THE INVENTION

The present invention is related to the industry of manufacturing hospital instruments. More specifically it is related to the industry of manufacturing instruments for extracting and draining fluids directly from a patient's bladder. Even more specifically it is related to the industry of manufacturing urinary catheters, which are made of biocompatible polymers, texturized on the surface with hydrophobic characteristics.

### BACKGROUND OF THE INVENTION

One of the most used medical apparatus today is catheters. Catheterization consists of the use of a catheter, which is a hollow tube with one or more lumens, being introduced into a cavity or conduit of the body to explore, extract or introduce substances. There are various kinds of catheters adapted to various uses and the material with which they are made is often biocompatible polymers. The most frequently used catheters are the urinary catheter, nasogastric tube, percutaneous endoscopic gastrostomy (PEG), rectal probe, oxygen probe and bronchial probe, however bladder catheterization is one of the most frequently used invasive procedures in hospitalization, since it is used on 15%-25% of hospitalized patients in order to monitor urine output or to empty the bladder [1].

Since the first urinary catheters a great variety of materials have been used in their design and manufacturing. An early overview reveals that since 3000 A.C. there is recorded intermittent use of urinary catheters when they were mode of capper, tin, bronze and gold [2, 3]. In the year 100 A.C., the Chinese used hollow onion stalks, dry sugar cane tubes and palm tree leaves treated with linseed oil, sundried and later varnished with lacquer.

The first malleable, elastic rubber for catheters was created by the French jeweler and goldsmith, Bernard, in 1779 [3]. A development of this design, a retention balloon probe, was developed in 1853, using rubber or woven cloth, soaked in cooked linseed oil [4]. The modern equivalent, originally made of latex and known as the Foley Catheter, was introduced for the first time in the mid 1930s, by Dr. Frederick B. Foley [5]. Today it is one of the most commonly used devices for the treatment of urinary incontinence (IU) by catheterization [6].

The typical Foley catheter comprises a tube OF axis with a primary lumen which is the conduit for the extraction, draining or insertion of fluids. Within this primary lumen is at least one other secondary lumen of a lesser caliber which serves as a conduit for the injection of gas or fluid for inflating a balloon which anchors the catheter within the patient's body. The anchoring balloon generally consists of a thin layer of elastic material near the tip and is connected on the edges to the surface of the catheter, An opening in wall of the tube allows the inflating gas or a fluid to pass under the elastic material and expand it until it is balloon shaped.

In practice, the Foley catheter is inserted inside the body cavity and a fluid is pumped into it through the secondary lumen in order to expand the anchorage balloon. This prevents accidental removal of the catheter from the patient besides holding the catheter in the correct position for its efficient use. Once the catheter is fixed, fluids may be drained from the body or therapeutic fluids may be injected into the body through the primary lumen. When the catheter is no longer necessary, the anchorage balloon is deflated by draining the inflating gas or fluid and the catheter is removed from the body. Although this basic design of the Foley Catheter has been used for many years, there are still various problems with its design. one of the most frequent of which is the infections related to its use.

Treatments which involve some kind of catheter are widely related to Healthcare Associated Infections (HAI). HAI are those infections which a patient acquires while receiving treatment for a medical condition or surgery and which had not manifested themselves nor was it in a period of incubation at the time the patient entered the institution. The infections are associated with various causes, such as post-operative complications, transmission between patients and healthcare workers and the use of medical devices [7].

Device-Associated infection (DAI) is an infection which shows up in a patient with an invasive device for example, a ventilator, a central catheter or a urinary catheter) which was used within 48 hours of the beginning of the infection. If the time period was over 48 hours, convincing evidence which indicates that the infection was associated with the use of the device must be present [8, 9]. Data from The Center for Disease Control and Prevention (CDC) reveals that the main types of infection which are present in hospitals are urinary tract infections, accounting for 34% of all infections [10, 11].

One of the problems normally encountered with the use of urinary catheters is the risk of infection. Various solutions for reducing this problem have been presented. The first solutions consisted of the use of polymeric materials with biocompatible silicon bases. This material let the catheter remain inside the body for a longer period of time and reduced the formation of incrustations of scale when it was used for extracting fluids from the bladder. However, the existence of bacterial infections associated with its use continues to exist in similar percentages to catheters or probes manufactured with other materials. Another solution to infections associated with the use of Foley Catheters is the use of antibacterial gel-based coatings. This solution consists of applying a lubricating coating to the catheter on the outside surface so that the introduction of the catheter into the conduits of the body are the least painful possible and at the same time to reduce the incidence of the growth of bacteria which cause infections.

One of the common problems with lubricated coatings with a high degree of lubricity, is the poor adhesion to the catheter besides the fact that it may alter the characteristics of the catheter such as it resistance, flexibility and easy manufacturing.

Oligodynamic metals such as silver in the form of ions or in small quantities are very effective bactericides. For some kinds of probes and catheters a polymeric sliver coating is used in the form of collagen which dissolves once the device is inside the patient's body. thus avoiding the growth of microorganisms. The use of oligodynamic metals in the form of fluid coatings presents the problem of the coating dissolving very quickly when in contact with bodily fluids and then the antibacterial protection no longer exists and in most cases the length of time the Foley Catheter is within the bladder duct of the patient is a prolonged period of time. Besides infections may enter the patient's body through the inside or outside of the catheter and not all catheters have coatings on both areas.

Still another kind of solutions is through the application of hydrophilic substances to the Foley Catheter in order to increase the adhesion of antibacterial substances and increase the action time. However, these coatings are often silicone gel-type coatings which become lubricants once they absorb water and dissolve very easily in the bodily fluids.

Still another kind of solution for incidents of bacterial infections and the formation of microbial films is the design of devices which are integrated into the protective barriers which prevent contamination of the catheter during its placement and which are thought to reduce the formation of colonies of bacteria and the resulting infection. These barriers are effective during the placement of the catheter inside the conduits of the body such as the urethra, but placement of the catheter in the urethra implies the natural mechanical seal of the urethral conduit remains open and thus contamination and the lodging of bacteria inside this and the tube of the catheter is a great probability.

Patents such as U.S. Pat. No. 3,598,127, U.S. Pat. No. 4,515,593, U.S. Pat. No. 4,575,371, U.S. Pat. No. 5,269,770, U.S. Pat. No. 0,326,639 A1 U.S. Pat. No. 0,198,195 A1, U.S. Pat. No. 0,146,680 A1, U.S. Pat. No. 0,319,325, U.S. Pat. No. 6,736,805 B2, U.S. Pat. No. 0,288,630 A2 and others present solutions to the problems of adhesion, the formation of bacterial films and infections related to the use of Foley Catheters such as those mentioned above.

As may be inferred from the text, the combination of all of these characteristics for the design of the Foley catheter offers only temporary solutions to the problems related to its use, but offers no permanent solutions.

The patent which we present claims the solutions to the needs for a) a Foley catheter made out of biocompatible polymeric material with long lasting characteristics in the body thus avoiding the formation of bacterial films and other incrustations, b) a Foley catheter that is texturized on the surface and which provides high hydrophobic properties and reduces the adhesion to tissues during long periods of contact and c) a Foley catheter which by way of its texturization reduces the incidence of bacterial infections associated with the use of this kind of medical devices.

### OBJECTIVES OF THE INVENTION

The main objective of the invention is to achieve a Foley catheter made of biocompatible polymeric material with a texturized surface which provides properties of high hydrophobicity for the reduction of the incidence of the formation of bacterial films and associated infections, when it is in contact with a patient's body.

Another of the objectives is to achieve a Foley catheter with a hydrophobic texturized surface which by way of this characteristic reduces the adhesion to body tissue when it is kept in contact for a long period of time.

Still another objective is to achieve a Foley catheter with a hydrophobic texturized surface which reduces the adhesion and formation of scale or other incrustations which cause infection and pain to the patient during the use of this medical device.

And all of those objective and advantages which shall become apparent with a reading accompanied by figures with illustrative, non-limiting, ends and which form an integral part of the present description.

### BRIEF DESCRIPTION OF THE INVENTION

The present invention refers to a catheter for draining and injecting bodily fluids of a patient, particularly it refers to a Foley type catheter for draining the fluids from inside the bladder of a patient during surgery or medical treatments. The material of the Foley catheter is a biocompatible polymeric material, and the catheter has embossed texturizing which provides properties of high hydrophobicity. The texturizing is formed during manufacturing by means of extrusion and injection. The hydrophobic texturization prevents the formation of bacterial films between the tubing and the patient's urethra and thus diminishes the incidence of bacterial infections and the adhesion of the tube to the walls of the urethra.

Moreover, the material of which the catheter is made has a silver-based coating which prevents the growth of infectious agents in the area of placement of the catheter and other problems related to the use of this device,

The Foley catheter with the hydrophobic texturized surface with low microbial adhesion of the present invention comprises a tube or axis with a first lumen, made of biocompatible polymeric material, which is the conduit for the extraction, draining or insertion of fluids, this primary tube present on its surface embossed texturization in patters and arrangements calculated to create the property of high hydorphobicity.

Within the first lumen and adhered to its wall there is at least one secondary lumen of lesser caliber which serves as a conduit for injecting an appropriate gas or fluid to inflate a balloon which anchors the catheter inside the patient's body.

The anchorage balloon consists generally of a thin layer of biocompatible elastic polymeric material which extends on the inside and around the catheter, proximal to the tip and connects by means of its edges to the texturized surface of the catheter. An opening in the wall, which only perforates the second lumen and is located below the thin film of elastic material, allows the inflating gas or fluid to enter below the thin layer of elastic material and this expands until it is balloon shaped. In the area proximal to the tip and in front of the anchorage balloon a small opening is formed in the texturized tubing which completely crosses through the first lumen and acts to block the passage of the fluids into the first lumen and its posterior draining. The tip of the tube is formed, by thermal means so that it makes a rounded tip and closes the first lumen at this end.

At the distal end of the tip of the tube is attached a collection funnel or comportment which may have at least two lines, depending on the number of sectioned lumens for the tube which may also be at least two lumens. One of the exits of the connecting funnel acts as an exit for the fluids drained from the patient's bladder while the other exit from the collection funnel acts as a valve to inflate the anchorage balloon.

In practice, the Foley catheter is inserted inside a body cavity, specifically from the urethra to the bladder, and an inflating fluid is pumped through the secondary lumen to inflate the anchorage balloon. This prevents accidental removal of the catheter from the patient besides placing the catheter in the correct position for its efficient use. Once the catheter is fixed in place, bodily fluids may be drained or therapeutic fluids may be injected into the body of the bladder or other conduits through the primary lumen. When the catheter is no longer needed, the anchorage balloon is deflated by means of draining the inflating gas or fluid and the catheter is removed from the body.

The main function of the texturization is to create the property of high hydorphobicity on the outside surface of the tube of the catheter, so that it prevents the formation of bacterial films and infections associated with them. The texturizing consists of circular base with a series of embossed geometric bodies, between 1 and 30 microns in size ordered in patterns or arrangements calculated to create a property of high hydrophobicity.

The texturized tubing is covered with a silver base forming a coating which also prevents the growth of bacteria on the surface of the catheter when it is in contact with the patient's urethra. This coating permanently adheres to the surface of the catheter tube and its effect lasts for the lifetime of the catheter.

### BRIEF DESCRIPTION OF THE FIGURES

FIG. 1 shows an isometric view of the hydrophobic texturized Foley catheter and its main components.

FIG. 2 shows a cross-sectional view of the tube of the hydrophobic texturized Foley catheter, indicating its sections of tube base, texturization, coating and lumens.

FIG. 3 shows a detailed section view of the anchorage balloon of the hydrophobic texturized Foley catheter.

FIG. 4 shows a detailed view of the texturization of the tube of the Foley catheter and patterns and arrangements of the geometric bodies on a circular base which comprise it.

### DETAILED DESCRIPTION OF THE INVENTION

For the detailed description, we shall rely on the figures of the preferred modality of the present invention.

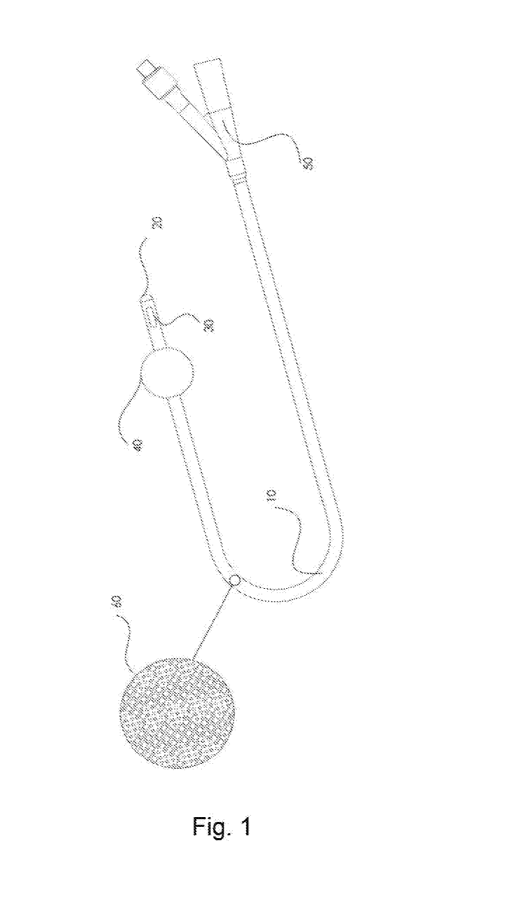

An isometric view of the hydrophobic texturized Foley catheter and ifs main components are shown in FIG. 1. The hydrophobic texturized Foley catheter with low microbial adhesion of the present invention consists of a tube or axis (10), between 100 and 400 mm in length with an exterior diameter ranging from between 1 and 30 mm. The tube or axis (10) has a primary lumen (11), with a diameter always less than the diameter of the end of the tube (10). The tube or axis (10) is made of biocompatible polymeric material. The first lumen (11) is the conduit for the extraction, draining or insertion of fluids. The tube (10) has a texturized embossed surface (60) in patterns or arrangements of circular based geometric bodies (61) calculated to create a property of high hydrophobicity.

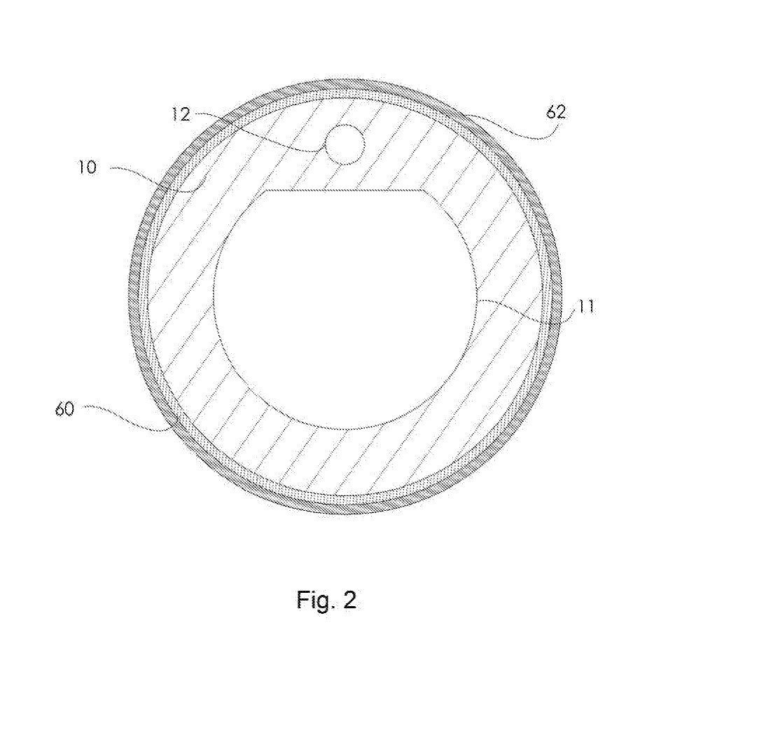

A cross-sectional view of the tube of the hydrophobic texturized Foley catheter is shown in FIG. 2. Within the primary lumen (11) and attached to its wall there is at least one other secondary lumen (12) with a smaller diameter of between 0.1 and 3 mm which acts as a conduit for injecting the appropriate gas or fluid to inflate a balloon (40) which anchors the catheter within the patient's body. The already texturized tube (10) is covered with a silver-based compound forming a coating (62) which also prevents the growth of bacteria on the surface of the catheter when it is in contact with the patient's urethra. This coating (62) permanently adheres to the surface of the tube of the catheter and its effect lasts for the lifetime of the Foley catheter.

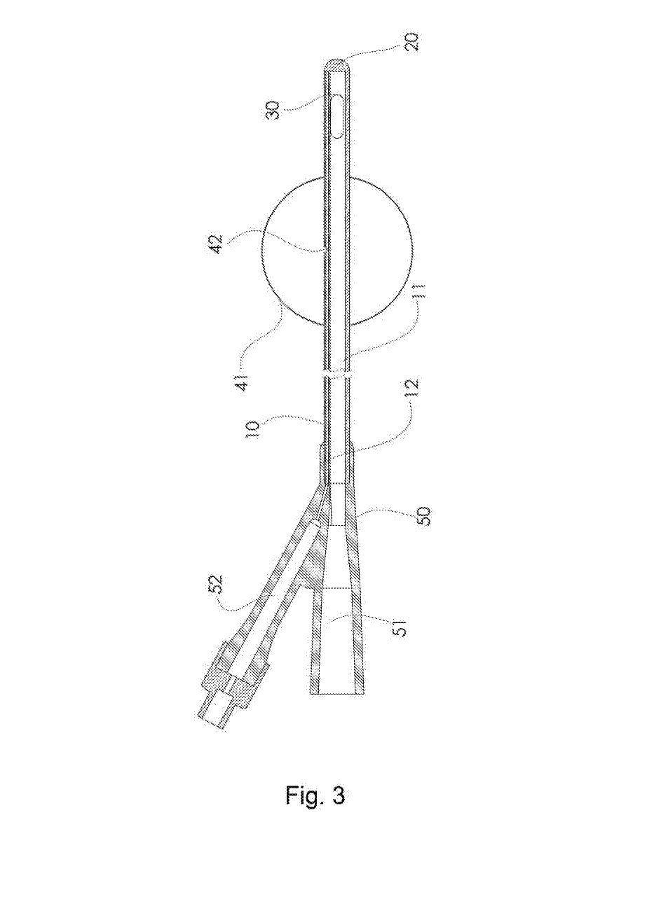

A detailed section view of the Foley catheter and of the anchorage balloon is shown in FIG. 3, The anchorage balloon (40) generally consists of a thin elastic layer (41) of biocompatible polymeric material which extends from outside and around the tube or axis (10), proximal to the tip (20) and is connected by means of its edges to the texturized surface (60) of the tube (10). An inflation opening (42) in the wall of the tube (10). which only perforates the secondary lumen (12) and is located below the thin layer of elastic material (41) and expands to the latter to create a balloon shape (40). In the area proximal to the tip (20) and in front of the anchorage balloon (40) another drainage opening (30) is formed in the texturized tube (10) which completely crosses through the first lumen (11) and which serves as a passageway for fluids into the first lumen (11) and its later drainage through the primary exit (51). The tip of the tube (20) is formed, using thermal means., so that it creates a rounded point and closes the tube (10) and its lumen (11) and lumen (12) at this end.

At the distal end of the tip (20) of the tube (10) is connected a collection funnel or compartment (50) which may have at least two lines depending on the number of lumens chosen for the tube (10) which may have at least two lumens. The primary exit (51) of the connecting funnel (50) acts as an exit for fluids drained from the patient's bladder while the secondary exit (52) of the collection funnel acts as an inflation valve for the anchorage balloon (40).

In practice the Foley catheter is inserted, by means of its tube (10) inside a body cavity, specifically inside the urethra to the bladder, and a fluid is pumped in through the secondary lumen (12) to expand the anchorage balloon (40). This prevents the accidental removal of the tube (10) of the catheter in the patient besides placing the tube (10) of the catheter in the correct position for its efficient use. Once the catheter, by means of the tube (10) is fixed, fluids from the body may be drained or therapeutic fluids may be injected into the body to the bladder and other conduits through the primary lumen (11). When the catheter is no longer needed, the anchorage balloon (40) is deflated by means of draining the inflating gas Or fluid and the tube (10) of the catheter is removed from the body.

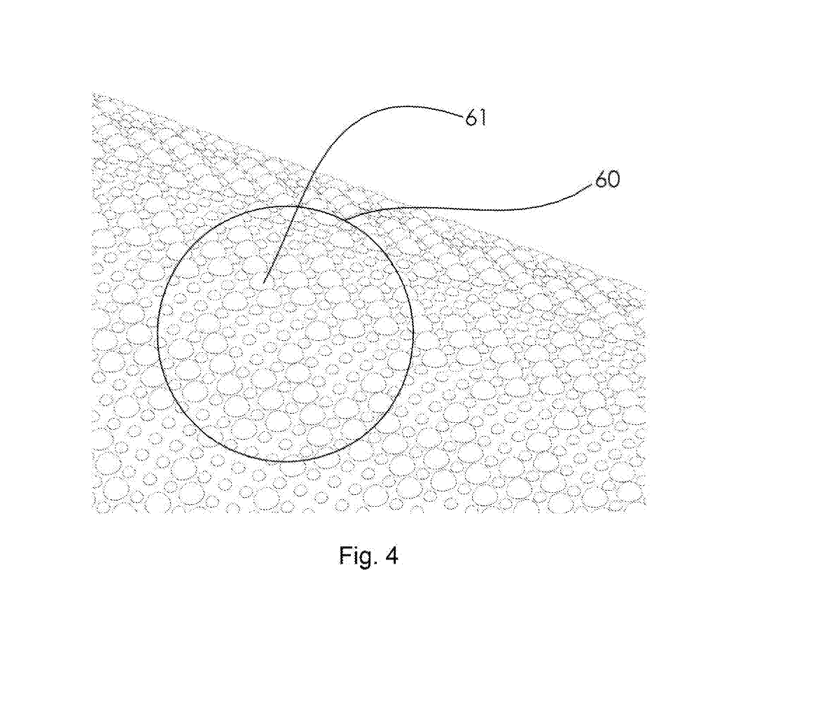

A detailed view of the texturizing with its patterns and arrangements in the tube of the catheter and the geometric bodies on the circular base which makes it up are shown in FIG. 4. The main function of the texturizing (60) is to create a property of high hydrophobicity or the external surface of the tube (10) of the catheter, so that it prevents the formation of bacterial films and infections associated with the latter. The texturizing (60) consists of a series of embossed geometric bodies on a spherical base (61), with a diameter of between 1 and 30 microns ordered in geometries calculated to create a high hydrophobic property.

### REFERENCES

[1] Dunn, S., Best Practice: Management of short term indwelling urethral catheters to prevent urinary tract infections, Institute Joanna Brings, Adelaide, 2000, No.6 Pp. 1-6, ISSN 1329-1874.

[2] G. M. Bruirisma, H. C. Van der Mei, H. J. Busscher, Bacterial adhesion to surface hydrophilic and hydrophobic contact lenses, J. Biomaterials, Elsevier, 22 (2001) 3217-3224.

[3] Seoktae Kong, Heechui Choi, Effect of surface hydrophobicity on the adhesion of S. cerevisiae onto modified surfaces by polystyrene-ran-suifonic acid) ramdom copolymer. Biointerf aces, Elsevier, 46 (2005) 70-77.

[4] Nuno Cerca, et at, Quantitative analysis of adhesion and biofilm formation or hydrophilic and hydrophobic surfaces of clinical isolates of Sstaphylococcus epidermidis. Research in Microbiology, Elsevier, 156 (2005) 506-514.

[5] Etsion I, Kligerman Y, Halperin G. Analytical and experimental investigation of laser-textured mechanical seal faces. Tribology Transactions 1999; 42: 511-6.

[6] Nakano M, Miyake K, Korenaga A, Sasaki S. Ando Y. Tribological properties of patterned NiFe-covered Si surfaces. Tribology Letters 2009; 35:133-9.

[7] Presidencia de la República de Colombia. Ministerio de la Protección Social. Decree 3518 Oct. 9, 2006. Available at:

http://www.ins.gov.co/lineas-de-accion/Subdireccion

[8] McKibben, L., T. Horan, J. I. Tokars et al. 2005. Guidance on Public Reporting of Healthcare-Associated Infections: Recommendations of the healthcare Infection Control Practices Advisory Committee. American journal of Infection Control 33 (4): 217-26.

[9] Horan, T. C. and T. G. Emori. 1997. Definitions of Key Terms Used in the NNIS System. American Journal of Infection Control 25 (2): 112-6.

[10] Department of health and human services. Action Plan to Prevent Healthcare-associated infections. 2009. Available at:

http://www.hhs.gov/ash/initiatives/hai/actionplan/hhs_hai_action_plan_final_06222009.pdfw

[11] World Health Organization. Patient Safety Programme. Geneva, Switzerland. Report on the Burden of Endemic Health Core-Associated Infection Worldwide. 2011.

The invention has been sufficiently described so that a person with average knowledge of the subject may reproduce and obtain the results mentioned in the present invention. However, any person knowledgeable in the field of the technique of the present invention may be able to make modifications riot described in the present application, however, if for the application of these modifications in a determined structure or manufacturing process of the same, the material claimed in the following claims is required, said structures are considered to be within the scope of the invention.

What is claimed is:

1. A Foley type catheter manufactured from biocompatible polymeric material characterized by the tube having a texturized embossed surface to provide it with hydrophobicity and prevent the first phase of an infection, which consists of fixing the microbe to the surface.

2. A Foley type catheter, as described in the previous claim characterized by the texturizing consisting of geometric bodies on a circular base, and distributed in geometric arrangements which provide the tube with the hydrophobic property.

3. A Foley type catheter, as described in the previous claims, also characterized by the tube (10) being in a range of between 100 and 400 mm long and with an external diameter in the range of 1 to 30 mm.

4. A Foley type catheter, as described in the previous claim, also characterized by the embossed texturizing (60) having a maximum height of 30 microns with separation between the geometric bodies on the spherical base (61) in the range of 1 to 20 microns.

5. A Foley type catheter, as described in claim 1, characterized by the distal end of the tip (20) of the tube (10) being connected to a collection funnel or compartment (50) with at least two lines.

6. A Foley type catheter, as described in claims 1 through 4, also characterized by the texturizing having a covering of a silver-based compound forming the coating.
